(19) United States
(12) Patent Application Publication
(10) Pub. No.: US 2021/0267748 A1
(43) Pub. Date: Sep. 2, 2021

(54) TRIFURCATED STENT GRAFT

(71) Applicant: Medtronic Vascular, Inc., Santa Rosa, CA (US)

(72) Inventors: Timothy BERTINI, Santa Rosa, CA (US); Haley KING, Santa Rosa, CA (US); Ian BARANOWSKI, Santa Rosa, CA (US)

(21) Appl. No.: 17/189,825

(22) Filed: Mar. 2, 2021

Related U.S. Application Data
(60) Provisional application No. 62/984,153, filed on Mar. 2, 2020.

(51) Int. Cl. A61F 2/07

(52) U.S. Cl. CPC A61F 2/07; A61F 2002/065

(57) ABSTRACT

Stent graft devices are disclosed that include a trifurcation of a main body into three branch lumens. The devices may be used to treat thoracoabdominal aortic aneurysms. The three branch lumens may split at a common point or plane in the device. One or more of the branch lumens may further split, such as bifurcate, in order to perfuse branch vessels of the aorta.

### CROSS-REFERENCE TO RELATED APPLICATIONS

This application claims the benefit of U.S. provisional application Ser. No. 62/984,153 filed Mar. 2, 2020, the disclosure of which is hereby incorporated in its entirety by reference herein.

### TECHNICAL FIELD

The present disclosure is generally related to stent grafts for endovascular treatment of aortic disease.

### BACKGROUND

Aneurysms occur in blood vessels in various locations due to age, disease, or genetic disposition, and insufficient blood vessel strength or resiliency may cause blood vessel walls to weaken and lose shape as the blood flows through the weakened blood vessels. Left untreated, these weakened blood vessels may continue to expand to the point where the blood vessel wall cannot hold, and the blood vessel may fail at the weakened locations, which may result in fatal consequences.

Many implantable medical devices are used and advantageously inserted to prevent rupture of an aneurysm. For example, a stent graft may be introduced, deployed and secured in a location with the blood vessel such that the stent graft spans the weakened areas of the blood vessel. The outer wall of the stent graft may abut and seal against the interior wall of the blood vessel to assist in channeling the blood flow to reduce any stress to the walls of the blood vessel at the weakened location.

Aneurysms may occur in a variety of locations within the aorta, some of which may not be treatable by conventional stent grafts. For example, conventional stent grafts may block branch arteries, which may result in inadequate blood flow to the associated parts of the body. Accordingly, there is a need for a system, a device, a method and/or an apparatus for a stent device with multiple branch or bypass lumens.

### SUMMARY

In an embodiment, a trifurcated stent graft includes a main body lumen extending along a longitudinal axis and having a proximal end and a distal end, and trifurcated limb gates extending from the distal end of the main body lumen. The trifurcated limb gates include: a first limb gate extending from the distal end of the main body, the first limb gate having a proximal end, a distal end, and a first wall extending therebetween that defines a first branch lumen therein; a second limb gate extending from the distal end of the main body, the second limb gate having a proximal end, a distal end, and a second wall extending therebetween that defines a second branch lumen therein; and a bypass gate extending from the distal end of the main body, the bypass gate having a proximal end, a distal end, and a third wall extending therebetween that defines a bypass lumen therein. The first wall, the second wall, and the third wall intersect at and branch from a trifurcation point.

In an embodiment, a trifurcated stent includes a main body extending along a longitudinal axis when the trifurcated stent graft is in a preinstalled configuration prior to insertion into a body of a patient, the main body having a proximal end and a distal end, the main body defining a first cross-sectional area at the distal end thereof. The trifurcated stent also includes trifurcated limb gates each having a proximal end extending from the distal end of the main body lumen. The trifurcated limb gates include: a first limb gate having a first wall defining a first branch lumen therein; a second limb gate having a second wall defining a second branch lumen therein; and a bypass gate having a third wall defining a bypass lumen therein. The first wall, the second wall, and the third wall combine to define a second cross-sectional area at the proximal end of the trifurcated limb gates, and the second cross-sectional area is equivalent to the first cross-sectional area.

In an embodiment, a stent graft includes a main body extending along a longitudinal axis and having a proximal end and a distal end, and exactly three limbs extending from the distal end of the main body. The three limbs include: a first wall defining a first branch lumen therein; a second wall defining a second branch lumen therein; and a third wall defining a bypass lumen therein. The first wall, the second wall, and the third wall intersect at a single trifurcation point.

### BRIEF DESCRIPTION OF THE DRAWINGS

FIG. 1 is a front view of a trifurcated stent graft, according to an embodiment.

FIG. 2 is a side view of the trifurcated stent graft of FIG. 1.

FIG. 3 is a schematic top view of a trifurcated stent graft, according to an embodiment.

FIG. 4 is a schematic cross-sectional view of the trifurcated stent graft, taken along line A-A of FIG. 1, according to an embodiment.

FIG. 5 is a bottom view of a trifurcated stent graft, according to an embodiment.

FIG. 6 is another front view of a trifurcated stent graft according to an embodiment.

FIG. 7 is top view of the trifurcated stent graft of FIG. 6, according to an embodiment.

### DETAILED DESCRIPTION

Embodiments of the present disclosure are described herein. It is to be understood, however, that the disclosed embodiments are merely examples and other embodiments may take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures may be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface which faces toward the axis. Terms such as “radial,” “axial,” “diameter,” “circumference,” etc. also are relative to the central axis. For example, the something extending along an “axial direction” means extending generally along or parallel to the central axis. The terms “front,” “rear,” “upper” and “lower” designate directions in the drawings to which reference is made.

Disclosed herein are systems, devices, methods and/or apparatuses for a stent device. The stent device may be formed as a single unitary body that has a main body lumen, a bypass lumen and one or more branch lumens. The stent device may be inserted into the aorta and the one or more branch lumens may be connected to or otherwise coupled with one or more extension grafts that are inserted into one or more branch vessels such as the celiac artery, the superior mesenteric artery, the right renal artery and/or the left renal artery. As a single unitary body, the main body lumen, the bypass lumen and the one or more branch lumens are integrally formed as a single piece that is deployed in an already complete state. This may be in contrast to modular device deployments where a first device is deployed in the patient and then a secondary device is deployed in the patient that couples or connects to the first device (e.g., like the extension grafts discussed, below). The terms unitary body and integrally formed may mean that the various portions are permanently connected together, such as by stitching. This reduces the amount of coverage length of the stent device and the number of failure points in comparison to a stent device that is modular.

As used herein, the proximal end of a prosthesis such as bifurcated stent graft is the end closest to the heart via the path of blood flow, whereas the distal end is the end furthest away (e.g., downstream of blood flow) from the heart during deployment. In contrast, the distal end of the catheter is usually identified to the end that is farthest from the operator (handle) while the proximal end of the catheter is the end nearest the operator (handle). However, those of skill in the art will understand that depending upon the access location, the stent graft and delivery system description may be consistent or opposite in actual usage.

The following detailed description is merely exemplary in nature and is not intended to limit the invention or the application and uses of the invention. Although the description is in the context of treatment of blood vessels such as the aorta, the teachings of this disclosure may also be used in any other body passageways where it is deemed useful, such as the coronary, carotid and renal arteries, etc.

Endovascular stent grafting, or endovascular aneurysm repair (EVAR), is a form of treatment for abdominal or thoracic aortic aneurysm that is less invasive than open surgery. Endovascular stent grafting uses an endovascular stent graft to reinforce the wall of the aorta and to help keep the damaged area from rupturing by excluding the aneurysm from blood flow and pressure. Stent grafts are generally tubular open-ended structures providing support for damaged, collapsing, or occluded blood vessels, such as the aorta. Stent grafts are flexible, which allows them to be inserted through, and conform to, tortuous pathways in the blood vessels. For example, stent grafts may be radially expandable from a radially-compressed (or radially-constricted) configuration for delivery to the affected vessel site (also referred to as a preinstalled configuration) to a radially-expanded configuration when deployed at the affected vessel treatment site, with the radially-expanded configuration having a larger diameter than the radially-compressed configuration. Stent grafts may be inserted in the radially compressed configuration and expanded to the radially-expanded configuration either through a self-expanding mechanism, or through the use of a balloon catheter, for example.

In one example, an EVAR procedure may include inserting a guide wire into a portion of the patient's body, such as the femoral artery. Once the guidewire is inserted into the artery, it may be gently pushed toward the site of the aneurysm. A stent graft delivery system, which may include a catheter and stent graft, may be placed over the guidewire and inserted along the guidewire into the site of the aneurysm. The stent graft may be guided within the catheter in its radially-compressed configuration and to the site of the aneurysm. There may be radiopaque markers at a distal end of the stent graft delivery system or on the stent graft itself to allow the surgeon to guide the stent graft into the proper position. Once in proper position, the stent graft can be expanded from the radially-compressed configuration to the radially-expanded configuration. This can be done, for example, by pulling back a stent-graft cover, allowing the stent graft to expand due to its fabric being biased outwards. Once deployed into the radially-expanded configuration, the stent graft can be held in place with metallic hooks or stents. The catheter can then be removed, while the stent graft remains

Stent grafts and other devices disclosed herein may be used to treat similar disease states to the devices disclosed in U.S. application Ser. No. 16/446,403 filed on Jun. 19, 2019 and claiming priority to U.S. Application No. 62/686,879 filed on Jun. 19, 2018, the disclosures of which are hereby incorporated in their entirety herein. One of ordinary skill in the art will understand, based on the present disclosure, that aspects of these disclosures may be combined with the present disclosure to form further embodiments. In addition, delivery systems and procedures used/described in Ser. No. 16/446,403 may be used with the devices disclosed herein.

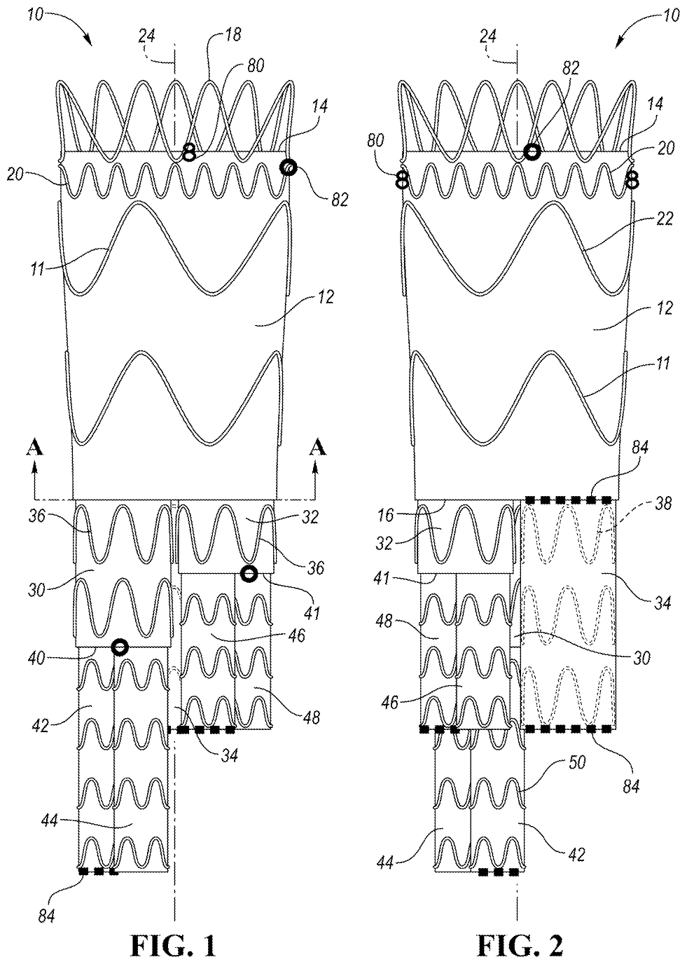

With reference to FIGS. 1 and 2, a trifurcated stent graft 10 is shown in its radially-expanded configuration. Once affixed within a blood vessel, the stent graft provides a tube or pipe for blood flow, guiding the blood flow through the stent graft 10. The stent graft 10 may be a single unitary body formed from various portions that when shaped by the stents form different lumens. The various portions may be in fluid communication with each other and formed together to form a single unitary body. A unitary body may have less failure points where the different portions of the graft may become disjointed, disconnected or otherwise fail to support a weakened portion of one or more blood vessels in comparison to a modular stent graft that has a modular tubular member with multiple portions fastened or otherwise connected together in the patient.

In embodiments, the graft material of the stent graft is non-permeable and can be constructed of woven textile or thin film, e.g., woven terephthalate (PET), thin film expanded polytetrafluoroethylene (ePTFE), or other non-permeable graft material. As graft material is non-permeable, blood or other fluid is prevented from passing through graft material. The stent graft 10 also has a plurality of stents 11. In embodiments, the stent(s) may be a made of any suitable material, such as a nitinol, stainless steel, nickel and/or titanium, and/or a bio compatible plastic. The stent may be composed of multiple rings and may be of any shape, such as a sinusoidal, zig-zag or v-shaped ring. The stent(s) may be sandwiched in between the layers of the graft and/or may be interwoven with the graft to form the stent graft.

The stent graft 10 may be referred to as a thoracoabdominal aortic aneurysm (TAAA) stent graft or a TAAA device. The stent graft 10 may have a proximal main body lumen 12 that is configured to be located in aorta and sealed therewith, for example, the descending aorta. The main body lumen 12 extends between a proximal end 14 and a distal end 16. The main body lumen 12 may include a proximal stent 18, which is the proximal-most stent of the stents 11 and may contact the aortic vessel wall and provide a radial force against the aortic vessel wall to anchor the TAAA device. In the embodiment shown, the proximal stent 18 is an open stent in which at least a majority of the proximal stent 18 is not covered by graft material; however, in other embodiments the proximal stent 18 is a closed stent in which at least a majority of the proximal stent 18 is covered by graft material. In at least one embodiment, the main body lumen 12 may be landed above (e.g., proximal to) the celiac artery. The main body lumen 12 may also include a sealing stent 20, which may be located at the proximal end 14 of the graft material of the main body lumen 12 to facilitate sealing of the proximal end 14 and avoid blood leaking between the stent graft 10 and the vessel wall. One or more body stents 22 may be located between the proximal end 14 and the distal end 16 of the main body lumen 12.

As will be explained in further detail, the main body lumen 12 of the stent graft may split into three branch lumens (e.g., trifurcate) at the distal end 16 of the main body lumen 12. In at least one embodiment, the branch lumens may trifurcate at a common location in the stent graft. Stated another way, the stent graft may go from one lumen (e.g., the main body lumen 12) to three lumens at a single plane that is perpendicular to the longitudinal axis of the main body (e.g., a plane that is perpendicular to blood flow). This may be in contrast to a stent graft where there is a series of bifurcations, one axially spaced from another, to form multiple branch lumens. Additional explanation of this structure and concepts are explained below.

The stent graft 10 may extend along a central longitudinal axis 24. The main body lumen 12 of the stent graft 10 extends axially in the distal direction until it terminates at the distal end 16 thereof. Thereafter, instead of the single lumen of the main body lumen 12, three branch lumens are present and extend in the axial direction. In other words, along a single plane (e.g., where line A-A is illustrated), the stent graft 10 transitions from a single-lumen design (e.g., the main body lumen 12) to a triple-lumen design; the stent graft trifurcates along a single plane.

The three branch lumens extending from the distal end 16 of the main body lumen 12 include a first branch lumen 30, a second branch lumen 32, and a third branch lumen 34. As will be further described below, each of the first branch lumen 30 and second branch lumen 32 may be configured to further bifurcate in order to perfuse side branch vessels of the aorta. Thus, these branch lumens 30, 32 may be referred to as a first limb gate 30 and a second limb gate 32, respectively. Each limb gate 30, 32 may include one or more stents 36. The third branch lumen 34 may be configured to allow perfusion of the distal aorta, and thus may be referred to as the bypass gate 34. The bypass gate 34 may include one or more stents 38. Due to the trifurcation, any fluid entering the main body lumen 12 of the stent graft 10 must exit through one of the limb gates 30, 32 or bypass gate 34. That is, the lumen of the main body lumen 12 is in fluid communication with the bypass gate 34 and the first and second limb gates 30, 32.

The limb gates 30, 32 as well as the bypass gate 34 may include a respective proximal end and a distal end. The proximal end of each limb gate 30, 32 and bypass gate 34 may be the distal end 16 of the main body lumen 12. The distal end 40 of the first limb gate 30 may define the end of the limb gate 30 and a beginning of bifurcated legs, namely a first leg 42 and a second leg 44. Likewise, the distal end 41 of the second limb gate 32 may define the end of the second limb gate 32 and a beginning of bifurcated legs, namely a third leg 46 and a fourth leg 48. Each of the legs 42, 44, 46, 48 may be provided with one or more stents 50. In the embodiment shown, stents 36 of the first and second limb gates 30, 32 as well as the stents 50 of legs 42, 44, 46, 48 are external stents (e.g., outside of graft material), while the stents in the bypass gate 34 are internal (e.g., inside of the graft material). However, it is to be understood that the stents 36, 50 can be any combination of internal and/or external.

In one embodiment, the first and second legs 42, 44 of the first limb gate 30 are configured to perfuse the two renal arteries, while the third and fourth legs 46, 48 of the second limb gate 32 are configured to perfuse the celiac and superior mesenteric arteries. This grouping may be based on the relative locations of the perfused arteries, with the renal arteries tending to be below (distal) the celiac artery and superior mesenteric artery (SMA). Therefore, as illustrated, the overall axial length of the first limb gate 30 may exceed the overall axial length of the second limb gate 32. However, it is to be understood that any of the legs 42, 44, 46, 48 may be used to perfuse any of the renal, celiac, and SMA arteries, or other arteries that branch from the aorta, and therefore the length of the first and second limb gates 30, 32 may be reversed from what is illustrated in FIGS. 1-2. In another embodiment, the first and second limb gates 30, 32 have equal axial lengths. Moreover, the lengths of the legs 42, 44, 46, 48 may differ from one another, and are not limited to the relative size shown in the Figures.

The legs 42, 44, 46, 48 and the bypass gate 34 may directly perfuse the target arteries described herein or they may perfuse them indirectly via branch extensions (e.g., tubular stent grafts). If configured to perfuse indirectly, one or more branch extensions may be coupled to the legs 42, 44, 46, 48 or the bypass gate 34 in order to reach the target vessel or area of the abdominal aorta. For the bypass gate 34, the branch extensions may further couple with additional devices in the distal aorta, such as a bifurcated abdominal aortic aneurysm (AAA) device (e.g., the Endurant devices sold by Medtronic, Inc.) or it may form a distal seal in the aorta.

The stent graft 10 may also include various radiopaque markers. The radiopaque markers may be sewn or otherwise attached to the graft material of the stent graft 10. The radiopaque markers may also be shaped or sized different from one another to signify to a surgical technician of a transition from one region of the stent graft to another region; the radiopaque markers provide orientation information to a surgical technician. For example, in the embodiment shown in FIGS. 1-2, a first type of radiopaque marker 80 and a second type of radiopaque marker 82 may be provided about the proximal end 14 of the main body lumen 12 in alternating fashion. The first type of radiopaque marker 80 may be a “figure-eight” shaped marker, and the second type of radiopaque marker 82 may be a circular shaped marker. The first and second types of radiopaque markers 80, 82 may be separated by about 90 degrees and alternating in type about the main body lumen 12 so that the surgical technician can be oriented to the right, left, posterior and anterior sides of the stent graft 10. The second type of radiopaque marker 82 may also be located on the stent material where the stent graft transitions from the first limb gate 30 to the first and second legs 42, 44, and where the stent graft transitions from the second limb gate 32 to the third and fourth legs 46, 48. Additional radiopaque markers (e.g., a third type of radiopaque marker 84) may be located at a proximal end of the bypass gate 34, a distal end of the bypass gate 34, and a distal end of at least some of the legs 42, 44, 46, 48. The locations of the radiopaque markers can vary from what is shown, and the illustrated example is merely a single one of many embodiments of such locations. Some of these radiopaque markers are also illustrated in FIG. 5, which is discussed below.

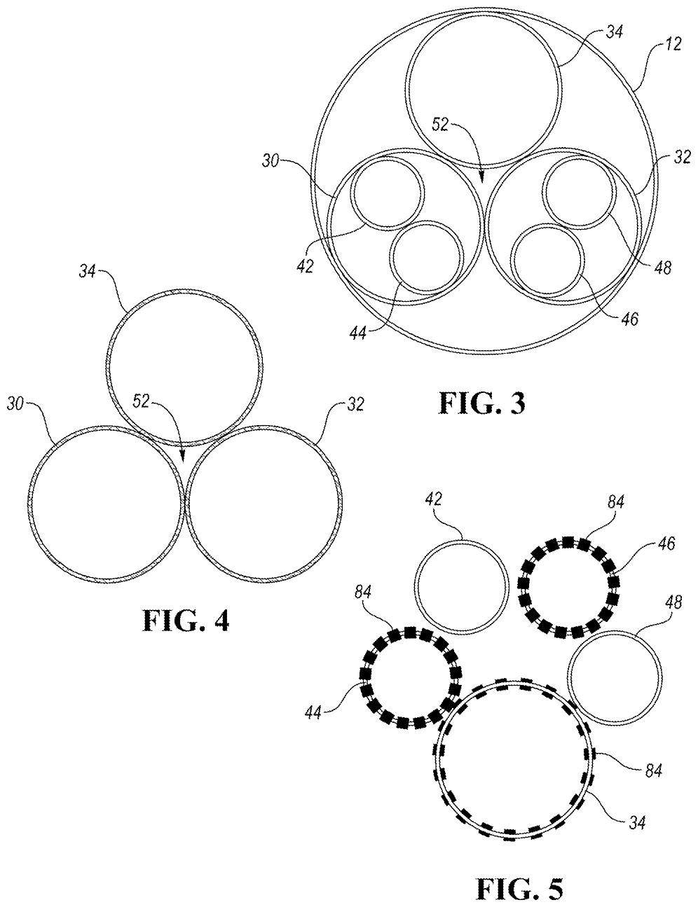

FIGS. 3-5 show various views of the stent graft 10 to illustrate the trifurcation. In particular, FIG. 3 shows a schematic top view of the stent graft 10 (e.g., looking down through the stent graft 10 from the top or proximal side 14 of the main body lumen 12). This schematic shows each of the three branch lumens—the first limb gate 30, the second limb gate 32, and the bypass gate 34. The first and second legs 42, 44 bifurcating from the first limb gate 30 are shown, as are the third and fourth legs 46, 48 bifurcating from the second limb gate 32.

It should be understood that this is a schematic representation; in embodiments, the overall outer diameter of the first limb gate 30 may be equivalent to the combined outer diameters of the first and second legs 42, 44. The same goes for the relationship between the second limb gate 32 and the third and fourth legs 42, 44.

FIG. 4 shows a schematic cross-sectional view taken along line A-A of FIG. 1 at the distal end 16 of the main body lumen 12 and looking in the upward or proximal direction, representing the plane in which the stent graft 10 transitions from a single lumen to three lumens. Since the view is taken looking up (e.g., in the proximal direction), the bifurcated legs of each limb gate 30, 32 are not visible. Again, this is a schematic representation. In both FIGS. 3 and 4, a central region 52 is illustrated as existing between the walls of the trifurcated branch lumens, but in actuality this region 52 may not exist. Instead, as will be described with reference to FIG. 7, the walls or edges that define a proximal portion of the trifurcated branch lumens may be shared and intersect at a single central point 54, which may be referred to as a point of trifurcation.

FIG. 5 shows a schematic bottom view of the stent graft 10 (e.g., looking up through the stent graft 10 from the bottom or distal side of the stent graft 10). This bottom view shows two sets of legs (e.g., legs 42, 44, 46, 48) and the bypass gate 34. The bypass gate 34 may have a larger diameter than any of the legs 42, 44, 46, 48. This is because the bypass gate 34 does not bifurcate, and is configured to provide perfusion of the distal aorta.

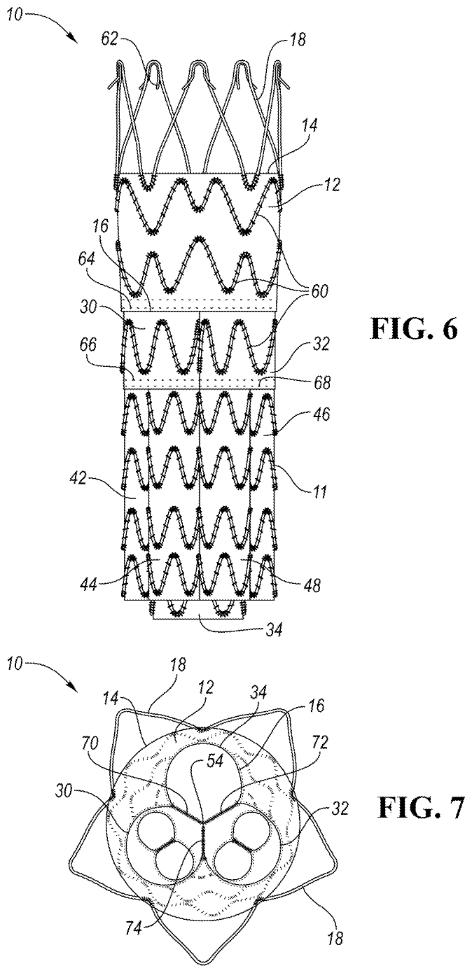

FIG. 6 is a front view of the stent graft 10, illustrating the stitching that may be present on the device. In this non-limiting example, stitching 60 may be provided to attach the stents 11 throughout the stent graft 10 to respective regions of the graft material. Stitching may also be provided to attach the proximal stent 18 to the graft material at or near the proximal end 14 of the main body lumen 12. As also shown in the Figure, the proximal stent 18 may be provided with active fixations 62 which may be configured to engage and/or penetrate the vessel wall in order to further anchor the stent graft (e.g., beyond radial force). In the embodiment shown, the active fixations 62 are in the form of barbs or tines extending outward at or near the apices of the stent. The active fixations 62 may be deployed or otherwise allowed to radially extend during deployment of the stent graft via a device within the stent graft delivery system. This is merely one example of active fixation and other types or specific configurations are contemplated herein.

Stitching may also be present to connect the various regions of graft material to form the overall stent graft 10. For example, stitching 64 may be present at or near the distal end of the main body lumen 12 to connect the main body lumen 12 to the trifurcated limb gates 30, 32, 34. As such, there may be some overlap in the material that makes up the limb gates 30, 32, 34 and the material that makes up the main body lumen 12. The distal end of the main body lumen can still be defined as the beginning edges of the trifurcated limb gates 30, 32, 34. Stitching 66 is also provided between the first limb gate 30 and the bifurcated legs 42, 44. Likewise, stitching 68 is provided to connect the second limb gate 32 and the bifurcated legs 46, 48. Similar overlap in the graft material may be present at the interface between the limb gates 30, 32 and the respective legs 42, 44, 46, 48.

The embodiment of FIG. 6 also illustrates an embodiment of the stent graft 10 in which the relative sizes of the trifurcated lumens 30, 32, 34 is different than in FIGS. 1-2. For example, in the embodiment of FIG. 6, the first limb gate 30 and the second limb gate 32 have identical lengths extending in the distal direction from the distal end 16 of the main body lumen 12. Also, the four legs 42, 44, 46, 48 have equal lengths. The bypass gate 34 has a longer length than the combined length of the first limb gate 30 and one of the legs 42, 44, and a longer length than the combined length of the second limb gate 32 and one of the legs 46, 48. Said another way, a distal end (e.g., bottom end) of the bypass gate 34 is the distal-most structure in the stent graft 10 in this embodiment.

FIG. 7 illustrates a top view of the stent graft 10, e.g., looking through the proximal end of the stent graft 10, according to an embodiment. As described previously, the trifurcation of the branch lumens 30, 32, 34 may occur at, or substantially at, a single plane. This plane may extend transverse to the central longitudinal axis 24 of the stent graft 10, or transverse to a central longitudinal axis of the main body lumen 12. The trifurcation may be achieved using any suitable method. In one example, the three branch lumens 30, 32, 34 may be individually and separately formed, and then attached at the trifurcation point 54. In another example, the trifurcated branch lumens 30, 32, 34 may be formed as a single integral piece during the textile manufacturing process. In another example, the trifurcated branch lumens 30, 32, 34 may be cut and sewn to form the trifurcated configuration. FIG. 7 illustrates such an embodiment, in which the branch lumens 30, 32, 34 are attached or joined together (e.g., sewn) such that each branch lumen 30, 32, 34 is connected to each of the other branch lumens 30, 32, 34 at a proximal end thereof.

As shown in FIG. 7, regardless of the manufacturing process, there may be a trifurcation point 54 or area that lies between and connects all three branch lumens 30, 32, 34. For example, each of the first limb gate 30, second limb gate 32, and bypass gate 34 are shown in FIG. 7 with the visible boundary lines representing the proximal edge of the wall of each gate that defines the respective lumen therein. While each of the limb gates 30, 32, and bypass gate 34 may have a respective outer wall to define a respective lumen therein, the outer walls may be joined together at the proximal edge thereof. For example, a portion of the proximal edge of the wall that defines the first limb gate 30 therein and a portion of the proximal edge of the wall that defines the bypass gate 34 are joined together at a first common edge 70. A portion of the proximal edge of the wall that defines the second limb gate 32 therein and a portion of the proximal edge of the wall that defines the bypass gate 34 therein are joined together at a second common edge 72. And, a portion of the proximal edge of the wall that defines the first limb gate 30 therein and a portion of the wall of the graft material that defines the second limb gate 32 therein are joined together at a third common edge 74. The three edges 70, 72, 74 may extend along a single plane (e.g., coplanar along plane A-A) and may intersect at the single trifurcation point 54.

In an embodiment, each limb gate 30, 32 and bypass gate 34 are at least partially joined to an adjacent one of the gates 30, 32, 34 via a common wall. Said another way, the edges 70, 72, 74 illustrated in FIG. 7 may extend in the distal direction to define common walls such that: a portion of the wall that defines the first limb gate 30 therein may also be the same material that defines a portion of the wall that defines the second limb gate 32 therein at 70; a portion of the wall that defines the second limb gate 32 therein may also be the same material that defines a portion of the wall that defines the bypass gate 34 therein at 72; and a portion of the wall that defines the bypass gate 32 therein may also be the same material that defines a portion of the wall that defines the first limb gate 30 therein at 74. In short, a single wall of graft material may separate each gates 30, 32, 34 from the other two adjacent gates. Each of these walls of graft material separating the gates 30, 32, 34 may be a single layer of graft material, or two layers of graft material joined together. The trifurcation point 54 and the lumen walls or edges 70, 72, 74 may be formed by and/or reinforced by stitching, as shown in FIG. 7.

In embodiments, a significant portion of the proximal end of each trifurcated lumen 30, 32, 34 may share a common wall or edge of an adjacent lumen 30, 32, 34. For example, in the embodiment shown in FIG. 7, each trifurcated lumen 30, 32, 34 shares between 10-20% of its circumference with one of the adjacent lumens 30, 32, 34. In other words, each of the shared edges or walls 70, 72, 74 can define between 10-20% of the circumference of a respective pair of lumens 30, 32, 34 that the shared edge or wall 70, 72, 74 is between. Taking the first lumen 30 as an example, the shared edge 70 may define 5-10% of the overall wall that defines the first lumen 30 therein, and the shared edge 74 may define another 5-10% of the overall wall that defines the first lumen 30 therein. In another embodiment, there are no shared walls or edges 70, 72, 74, and instead the three trifurcated lumens 30, 32, 34 intersect at only a common point 54.

The trifurcation point 54 may be an intersection of all common edges or walls that define a portion of the trifurcated lumens 30, 32, 34. In other words, the common edge or wall 70 that divides (and at least partially defines) the first limb gate and the bypass gate 34 intersects the common edge or wall 72 and the common edge or wall 74 at the trifurcation point 54.

As can be seen in FIG. 7, the main body lumen 12 of the stent graft 10 tapers slightly radially inward as the main body lumen 12 extends in the distal direction; the cross-sectional area of the main body lumen 12 at the proximal end 14 thereof is larger than the cross-sectional area of the main body lumen 12 at the distal end 16 thereof. Said another way, the diameter at the proximal end 14 exceeds the diameter at the distal end 16. In embodiments, the distal end 16 of the main body lumen 12 also defines the proximal end of the three trifurcated lumens 30, 32, 34. In other words, as the single tubular shape of the main body lumen 12 ends at its distal end 16 thereof, the three trifurcating lumens 30, 32, 34 begin. It can thus be said that the total cross-sectional area (or outer profile) of the three trifurcating gates 30, 32, 34 at the plane of trifurcation is equivalent to the cross-sectional area (or outer profile) of the main body lumen 12 immediately above (e.g., proximate) the trifurcation point 54.

While the trifurcation point 54 is shown at the center of the three gates 30, 32, 34, the trifurcation 54 may be offset from the center depending on the desired sizes and orientations of the gates 30, 32, 34 for various applications.

The trifurcation of the three lumens being at a single plane and/or a single point, as described herein, enables the individual gates 30, 32, 34 and respective legs 42, 44, 46, 48 to be located higher (e.g., more proximal) on the stent graft than previously possible. In other words, the main body lumen 12 can be shorter than in previous stent grafts. In an embodiment, the length of the main body lumen 12 is less than 50 millimeters (mm). In a more particular embodiment, the length of the main body lumen 12 is between 20 and 30 mm. A shorter length of material of the main body lumen 12 allows the main body lumen 12 to terminate more distally (lower) in the aorta, thus covering less of the segmental arteries which branch from the aorta and provide blood flow to the spine. This reduces the risk of spinal cord ischemia and all the complications that come with it. Covering less segmental arteries can also result in less type-II endoleaks, a condition in which blood flows into the aneurysm sac from these feeder vessels.

While not illustrated in this disclosure, the stitching that joins adjacent gates 30, 32, 34 may be made along a path that is oblique to the longitudinal axis 24. In other words, the proximal end of one or more of the trifurcating lumens 30, 32, 34 can be angled to extend in the distal direction outward from the single point 54 of trifurcation. This can relieve bunching up of the stitches when in the radially-compressed configuration. The teachings of providing such an oblique seam are disclosed in U.S. patent application Ser. No. 16/685,271 titled OBLIQUE SEAM FOR REDUCED STENT GRAFT PACKING DENSITY IN DELIVERY SYSTEM, which is hereby incorporated by reference in its entirety.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

The words used in the specification are words of description rather than limitation, and it is understood that various changes may be made without departing from the spirit and scope of the disclosure. As previously described, the features of various embodiments may be combined to form further embodiments that may not be explicitly described or illustrated. While various embodiments could have been described as providing advantages or being preferred over other embodiments or prior art implementations with respect to one or more desired characteristics, those of ordinary skill in the art recognize that one or more features or characteristics may be compromised to achieve desired overall system attributes, which depend on the specific application and implementation. As such, embodiments described as less desirable than other embodiments or prior art implementations with respect to one or more characteristics are not outside the scope of the disclosure and may be desirable for particular applications.

What is claimed is:

1. A trifurcated stent graft comprising:
a main body lumen extending along a longitudinal axis and having a proximal end and a distal end;
trifurcated limb gates extending from the distal end of the main body lumen, the trifurcated limb gates including:
a first limb gate extending from the distal end of the main body, the first limb gate having a proximal end, a distal end, and a first wall extending therebetween that defines a first branch lumen therein,
a second limb gate extending from the distal end of the main body, the second limb gate having a proximal end, a distal end, and a second wall extending therebetween that defines a second branch lumen therein, and
a bypass gate extending from the distal end of the main body, the bypass gate having a proximal end, a distal end, and a third wall extending therebetween that defines a bypass lumen therein,
wherein the first wall, the second wall, and the third wall intersect at and branch from a trifurcation point.

2. The trifurcated stent graft of claim 1, wherein the main body lumen transitions to the trifurcated limb gates along a trifurcation plane, and the trifurcation point is located on the trifurcation plane.

3. The trifurcated stent graft of claim 2, wherein the distal end of the main body lumen is located on the trifurcation plane, and wherein the proximal ends of the first limb gate, the second limb gate, and the bypass gate are located on the trifurcation plane.

4. The trifurcated stent graft of claim 3, wherein a cross-sectional area of the main body lumen is equivalent to a cross-sectional area of the trifurcated limb gates on the trifurcation plane.

5. The trifurcated stent graft of claim 1, wherein a first portion of the first wall and a first portion of the second wall define a first common wall that separates the first limb gate from the second limb gate.

6. The trifurcated stent graft of claim 5, wherein a second portion of the first wall and a first portion of the third wall define a second common wall that separates the first limb gate from the bypass limb gate.

7. The trifurcated stent graft of claim 6, wherein a second portion of the second wall and a second portion of the third wall define a third common wall that separates the second limb gate from the bypass limb gate.

8. The trifurcated stent graft of claim 7, wherein the first common wall, the second common wall, and the third common wall intersect at the trifurcation point.

9. The trifurcated stent graft of claim 1, further comprising:
first and second bifurcated legs extending from the distal end of the first limb gate; and
third and fourth bifurcated legs extending from the distal end of the second limb gate.

10. A trifurcated stent graft comprising:
a main body extending along a longitudinal axis when the trifurcated stent graft is in a preinstalled configuration prior to insertion into a body of a patient, the main body having a proximal end and a distal end, the main body defining a first cross-sectional area at the distal end thereof;
trifurcated limb gates each having a proximal end extending from the distal end of the main body lumen, the trifurcated limb gates including:
a first limb gate having a first wall defining a first branch lumen therein,
a second limb gate having a second wall defining a second branch lumen therein, and
a bypass gate having a third wall defining a bypass lumen therein,
wherein the first wall, the second wall, and the third wall combine to define a second cross-sectional area at the proximal end of the trifurcated limb gates, and wherein the second cross-sectional area is equivalent to the first cross-sectional area.

11. The trifurcated stent graft of claim 10, wherein the first wall, the second wall, and the third wall intersect at a trifurcation point.

12. The trifurcated stent graft of claim 11, wherein the trifurcation point is located along the longitudinal axis.

13. The trifurcated stent graft of claim 10, wherein:
a proximal edge of the first wall is joined with a proximal edge of the second wall to define a first common edge separating the first branch lumen from the second branch lumen;
the proximal edge of the second wall is joined with a proximal edge of the third wall to define a second common edge separating the second branch lumen from the bypass branch lumen;
the proximal edge of the third wall is joined with the proximal edge of the first wall to define a third common edge separating the bypass branch lumen from the first branch lumen.

14. The trifurcated stent graft of claim 13, wherein the first common edge, the second common edge, and the third common edge intersect at a trifurcation point.

15. The trifurcated stent graft of claim 14, wherein the first common edge, the second common edge, and the third common edge are all located on a common plane.

16. The trifurcated stent graft of claim 10, further comprising:
first and second bifurcated legs extending from a distal end of the first limb gate; and
third and fourth bifurcated legs extending from the distal end of the second limb gate.

17. A stent graft comprising:
a main body extending along a longitudinal axis and having a proximal end and a distal end;
exactly three limbs extending from the distal end of the main body, the three limbs including:
a first wall defining a first branch lumen therein,
a second wall defining a second branch lumen therein, and
a third wall defining a bypass lumen therein;
wherein the first wall, the second wall, and the third wall intersect at a single trifurcation point.

18. The stent graft of claim 17, wherein:
a proximal edge of the first wall is joined with a proximal edge of the second wall to define a first common edge separating the first branch lumen from the second branch lumen;
the proximal edge of the second wall is joined with a proximal edge of the third wall to define a second common edge separating the second branch lumen from the bypass branch lumen;
the proximal edge of the third wall is joined with the proximal edge of the first wall to define a third common edge separating the bypass branch lumen from the first branch lumen.

19. The stent graft of claim 18, wherein the first common edge, the second common edge, and the third common edge intersect at the single trifurcation point.

20. The stent graft of claim 19, wherein the first common edge, the second common edge, and the third common edge are all coplanar.
